(19) United States
(12) Patent Application Publication
(10) Pub. No.: US 2022/0168585 A1
(43) Pub. Date: Jun. 2, 2022

(54) Dynamically Controlling Self-Directed Magnetic Stimulation

(71) Applicant: THE METHODIST HOSPITAL, Houston, TX (US)

(72) Inventor: Santosh A. Helekar, Sugar Land, TX (US)

(21) Appl. No.: 17/434,646

(22) PCT Filed: Mar. 4, 2020
(86) PCT No.: PCT/US2020/020943

§ 371 Date: Aug. 27, 2021

Related U.S. Application Data
(60) Provisional application No. 62/813,425, filed on Mar. 4, 2019.

(51) Int. Cl. A61N 2/00; A61N 2/12

(52) U.S. Cl. CPC A61N 2/006; A61N 2/008; A61N 2/12

(57) ABSTRACT

To generate operational parameters for Transcranial Magnetic Stimulation (TMS), training data that includes indications of previously conducted TMS sessions is generated. A machine learning model is trained using the training data. The trained machine learning model is applied to one or more parameters related to an individual to generate operational parameters for a TMS session. A device for applying TMS to conduct a TMS session with the individual is operated in accordance with the generate operational parameters.

### CROSS-REFERENCE TO RELATED APPLICATIONS

The present application claims the priority benefit of U.S. Provisional Patent Application No. 62/813,425, filed Mar. 4, 2019, and entitled “DYNAMICALLY CONTROLLING Self-Directed MAGNETIC STIMULATION,” the disclosure of which is incorporated herein by reference in its entirety.

### FIELD OF THE DISCLOSURE

The present disclosure generally relates to controlling a medical procedure administered at a personal electronic device and, more particularly, to dynamically controlling and optimizing operational parameters of a device that controls Transcranial Magnetic Stimulation (TMS) therapy sessions.

### BACKGROUND

The background description provided herein is for the purpose of generally presenting the context of the disclosure. Work of the presently named inventors, to the extent it is described in the background section, as well as aspects of the description that may not otherwise qualify as prior art at the time of filing, are neither expressly nor impliedly admitted as prior art against the present disclosure.

Today, there are certain medical procedures that general-purposes computing devices, such as laptop computers, tablet computers, or smartphones, can implement by controlling specialized equipment according to a certain number of parameters. Moreover, the specialized equipment required for some of these procedures is portable, and the procedures accordingly can be administered both at a hospital and outside a hospital setting (e.g., at a patient's home).

For example, Transcranial Magnetic Stimulation (TMS) is a non-invasive procedure in which magnetic stimulation is applied to the brain in order to modify the natural electrical activity of the brain. More particularly, TMS involves applying a rapidly changing magnetic field to the brain of an individual to induce weak electric currents in the brain through electromagnetic induction. These weak electric currents modify the natural electrical activity of the brain.

Today, TMS can be used to provide therapy to an individual, assist in diagnosis, and/or to map out brain function in neuroscience research. Moreover, TMS has been approved by the Food and Drug Administration (FDA) for treating depression. TMS is also currently being investigated in the management of various other neurological and psychiatric disorders, including stroke, migraines, Parkinson's disease, tinnitus, autism, schizophrenia, etc.

Generally speaking, when administering TMS therapy and/or testing is to be applied to an individual, a clinician or investigator must determine the appropriate number and positioning of the stimulator(s) to be placed on the head of the patient or subject, as well as when the stimulators are to be turned on, at what rate/frequency, according to what pattern, and at what stimulus strength, to generate a changing magnetic field that is safe for the individual. These factors collectively can be referred to as “TMS parameters.” Currently, these TMS devices are operated using pre-set TMS parameters for various conditions.

However, multiple individuals with the same condition may require different TMS stimulus parameters for effective treatment. Currently, there are no effective and safe techniques for dynamically adjusting TMS parameters based on individual needs.

### SUMMARY

The present disclosure describes techniques for providing highly individualized Transcranial Magnetic Stimulation (TMS) therapy. More specifically, these techniques are related to generating specialized TMS stimulus parameters for a particular individual using a machine learning model trained with past TMS session data for this individual, test data such as encephalogram (EEG) readings, feedback regarding relief from pain and other symptoms provided by the individual, etc. As discussed in more detail below, a software application (“TMS application”) can execute on a client device such as a laptop computer, a tablet computer, a smartphone, etc. to train the machine learning model. The software application then can apply the output of the machine learning model to a device that activates a set of stimulators according to a particular pattern (referred to below as “TMS device”). In at least some of the implementations, the stimulators are miniaturized for use with a head mount by using rotating high field strength permanent magnets instead of electromagnetic coils, and accordingly can be referred to as “microstimulators.”

When training the machine learning model, the TMS application can receive, from a network server or another suitable source, initialization data that specifies initial operational parameters such as the initial locations of the microstimulators on the scalp, the speed at which motors in the microstimulators rotate the respective magnets, the duration of the stimulus period, the repetition rate of the stimulus period, etc. In some cases, the parameters can apply to individual microstimulators, so that for example a microstimulator placed on the side of the head generates a magnetic field of a different strength than a microstimulator placed near the crown. The TMS application can receive different sets of initial parameters for different configurations of the TMS device.

In some implementations, a network server or another suitable computing device determines the initial locations of the microstimulators on the scalp for the individual based on an EEG reading, a magnetic resonance imaging (MRI) scan, a positron emission tomography (PET) scan, etc. as well as the biometric measures of the individual's head. The initial locations of the microstimulators can be a set of (x,y) coordinates for a suitable projection of the scalp (e.g., the EEG electrode projection). Depending on the implementation, the network server can determine these parameters algorithmically or using machine learning or another suitable optimization technique. The network server similarly can determine the other initial operating parameters, as indicated below.

During training, the TMS application can obtain description of past TMS sessions for the individual and generate training data (e.g., feature vectors) based on this data. As discussed in more detail below, the training data can include parameters of TMS sessions (duration, frequencies, repetition rates, etc. used during the session) as well as indicators that can be used as labels (e.g., relief from chronic pain, decrease in tremors, or suppression of focal epileptic seizures at certain locations or in general). Moreover, the training data can include real-time EEG readings that indicate the effect of a particular TMS session on the electrical activity of the brain. The TMS application can apply the training data to the model using any suitable techniques, and the model can generate predictions in the form of operational parameters that are likely to yield positive user feedback and expected EEG readings.

To apply the output of the machine learning model to the TMS device, the TMS application in some implementations configures a controller operating in the TMS device with the operational parameters for the duration of the session. In other implementations, the TMS application dynamically adjusts at least some of the operational parameters based on real-time feedback. For example, the TMS application can dynamically adjust the speed of rotation of some or all of the motors. Moreover, the stimulators in some implementations are mounted on a motorized frame (e.g., an antero-posterior and medio-lateral frame with the vertex as the origin), and the TMS application can dynamically and automatically adjust the locations of the microstimulators during the session.

In some implementations, a network server receives data from multiple user devices that execute respective instances of the TMS application, preferably in an anonymized format and in compliance with the relevant privacy controls. The network server can train a model in a generally similar fashion using a larger training data set to generate operational parameters for TMS therapy. Unlike the operational parameters the TMS application can generate, the operational parameters generated at the network server are not specific to any individual. However, the network server can generate operational parameters for certain types of users and/or neuropsychiatric conditions. Referring back to the discussion of the machine learning model stored in a user device, a particular instance of the TMS application can receive these operational parameters as part of the initialization data, which the machine learning model subsequently adjusts to optimize these parameters for the particular individual.

Further, to increase operational safety, the TMS application can prevent the TMS device from operating on the same individual for excessive periods of time. The network server and/or the user device can provide these limits on a per-session basis (e.g., no more than X minutes of continuous use), a daily basis (e.g., no more than Y minutes of use per day), a weekly basis, etc. Still further, the TMS application can apply biometric verification, such as facial recognition or a fingerprint scan, to prevent individuals from improperly operating the TMS device.

More generally, the techniques for determining, tuning, and applying parameters for a therapy session, as well as for ensuring safety when conducting therapy sessions in the absence of professional supervision, can apply to other procedures and technologies. For example, portable devices can train and apply machine learning models for controlling an exoskeleton used in facilitating recovery of persons who suffered a spinal cord injury or a neurologic disease, a system for respiratory therapy, a massage system, etc.

One example embodiment of these techniques is a computer-implemented method for generating operational parameters for TMS. The method can be executed by processing hardware and includes generating training data that includes indications of previously conducted TMS sessions, training a machine learning model using the training data, applying the trained machine learning model one or more parameters related to an individual to generate operational parameters for a TMS session, and causing a system for applying TMS to conduct a TMS session with the individual in accordance with the generated operational parameters.

Another example embodiment of these techniques is a computing device comprising one or more processors, a user interface, an interface to couple the computing device to a device for applying TMS to an individual during a TMS session, and a computer-readable memory coupled to the one or more processors, the memory storing instructions that implement a method according to the method above.

Still another example embodiment of these techniques is a method in a computing device for controlling application of a TMS therapy to an individual. The method includes receiving, via a user interface, a request from a user to start TMS therapy; detecting a short-range communication link between the computing device and a controller of a device for applying TMS therapy to the user during a TMS session; verifying identity of the user; and transmitting, by the processing hardware via the short-range communication link, a set of instructions for the controller to conduct the TMS session in accordance with the verified identity of the user.

### BRIEF DESCRIPTION OF THE DRAWINGS

FIG. 1 is a block diagram of an exemplary system in which techniques for controlling the provision of Transcranial Magnetic Stimulation (TMS) therapy to an individual can be implemented.

FIG. 2 is a schematic view illustrating an example device for providing TMS to an individual.

FIG. 3 is a block diagram of an example machine learning model which the system of FIG. 1 can use to calculate stimulus parameters to be used in the provision of TMS therapy to an individual.

FIG. 4 illustrates an example user interface screen which a TMS application on a mobile device can generate for collecting user feedback associated with TMS therapy.

FIG. 5 illustrates a flow diagram of an example computer-implemented method of controlling the administering of TMS therapy to an individual.

FIG. 6 illustrates a flow diagram of an example computer-implemented method of training a TMS machine learning model customized for a particular individual.

FIG. 7 illustrates a flow diagram of an example computer-implemented method of training a non-individualized TMS machine learning model.

### DETAILED DESCRIPTION

As discussed below, the techniques of this disclosure allow computing devices to generate TMS stimulus parameters for a particular individual using a machine learning model, generate TMS stimulus parameters applicable to multiple individuals using another machine learning model, and improve operational safety of a Transcranial Magnetic Stimulation (TMS) device. Although the examples below relate primarily to TMS, at least some of the techniques of this disclosure also can apply to other systems in which a computing device controls a medical and/or a therapeutic procedure according to a set of adjustable parameters related to position, intensity, or frequency, for example.

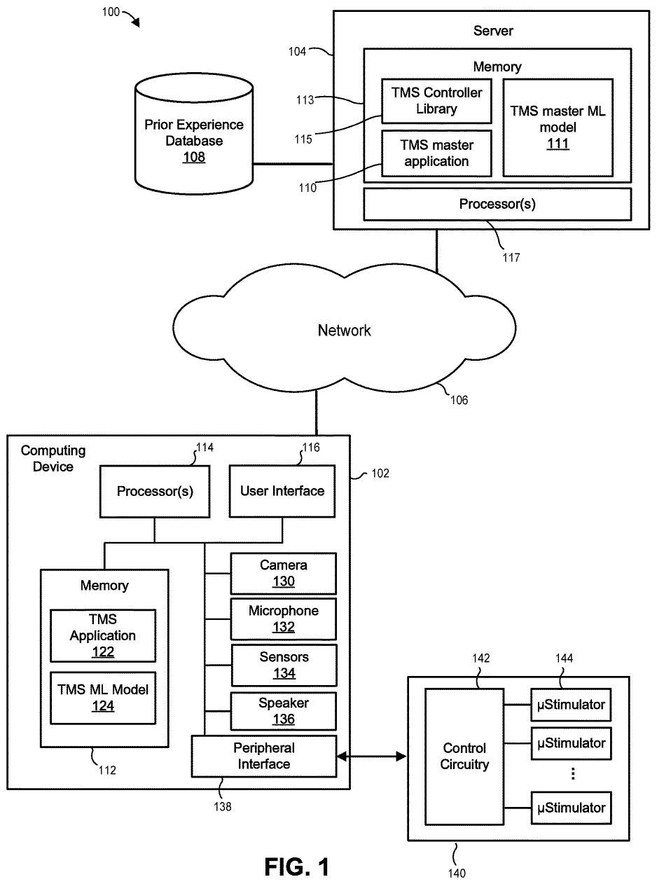

Referring to FIG. 1, an example computing environment 100 in which the techniques of this disclosure can be implemented includes a computing device 102 configured to communicate with a server 104 via a network 106, and further configured to transmit instructions to a TMS device or subsystem 140. The network 106 can be, for example, a local area network (LAN) or a wide area network (WAN) such as the Internet. The computing device 102 can communicate with the TMS device 140 via a short-range communication link, such as a wireless personal area network (WPAN) link, e.g., Bluetooth®.

The computing device 102 can be, for example, a personal computer, a portable device such as a tablet computer or smartphone, a wearable computing device, etc. As illustrated in FIG. 1, the computing device 102 can include processing hardware such as a memory 112 and one or more processors 114 (which may be, e.g., microcontrollers and/or microprocessors). The computing device 102 also can include a user interface 116, a camera 130, a microphone 132, one or more sensors 134, and a speaker 136, as well as a peripheral interface 138 (e.g., WPAN, WLAN, USB, infrared, etc.) for communicating with the TMS device 140.

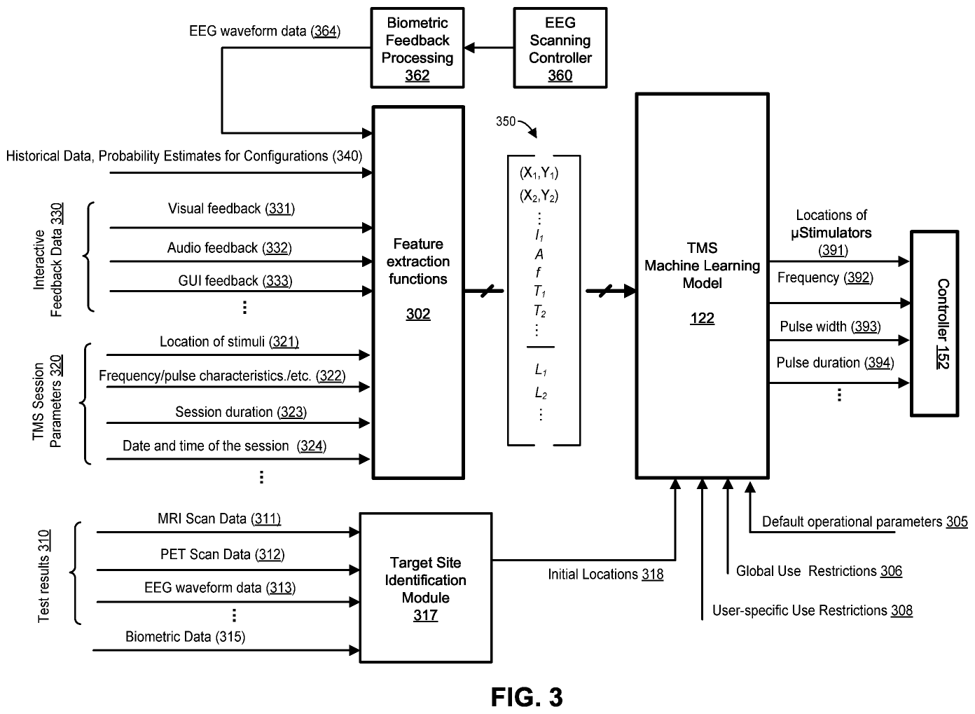

The memory 112 of the computing device 102 can be a non-transitory memory and can include one or several suitable memory modules, such as random access memory (RAM), read-only memory (ROM), flash memory, other types of persistent memory, etc. The memory 112 further includes a TMS application 122 and a TMS machine learning model 124. Generally speaking, the TMS application 122 can obtain user information and use this user information to train and operate the TMS machine learning model 124 in accordance with the scheme illustrated in FIG. 3. Based on the TMS stimulus parameters generated using the TMS machine learning model, the TMS application 122 transmits instructions for administering TMS therapy to the TMS device 140 via the peripheral interface 138. For example, the TMS application 122 can transmit instructions that cause the control circuitry 142 (or simply “controller 142”) to reposition microstimulators 144 of the TMS device, activate or deactivate microstimulators 144 of the TMS device in certain locations, change the frequency of magnet rotation, etc., as needed, in order to administer individualized TMS therapy to the user.

In some implementations, before transmitting the TMS instructions to the controller 140, the TMS application 122 verifies the identity of the user. In one example, the camera 130 captures an image of the face of the user, and the TMS application 122 compares the captured image of the face of the user to one or more stored images of the face of the user (e.g., using a facial recognition algorithm) to verify the identity of the user. In some implementations, the TMS applications invoke an appropriate application programming interface (API) to apply a suitable facial recognition algorithm, and the API in turn can transmit and receive information via the network 106. In this manner, the TMS application 122 can ensure that the user receives the correct individualized TMS treatments at the correct times.

Additionally, in some implementations, before transmitting the TMS instructions to the controller 140, the TMS application 122 determines whether the user has properly positioned the TMS device on his or her head for TMS treatment. In one example, the camera 130 captures an image of the user wearing the TMS device, and the TMS application 122 analyzes the image to determine whether the position of the TMS device on the user's head matches a recommended position of the TMS device for the type of TMS treatment that will be administered to the user. For instance, if the TMS device is improperly positioned on the user's head, the TMS application may cause a message to be displayed via the user interface 116 or verbally communicated via the speaker 136, with instructions to the user for correcting the positioning of the TMS device.

The TMS application 122 can store TMS session parameters (e.g., location of the microstimulators, frequency of rotation for the motors, pulse characteristics, session duration, date and time of session, etc.) each time TMS therapy is administered to the user. The TMS application 122 can use these stored TMS session parameters, along with user feedback data collected via the user interface 116, camera 130, microphone 132, and the one or more sensors 134 during or after each TMS treatment to train the TMS machine learning model 124. For example, the user feedback data may include data indicating headache and other side effects or relief from disease symptoms experienced by the user during or after each TMS treatment.

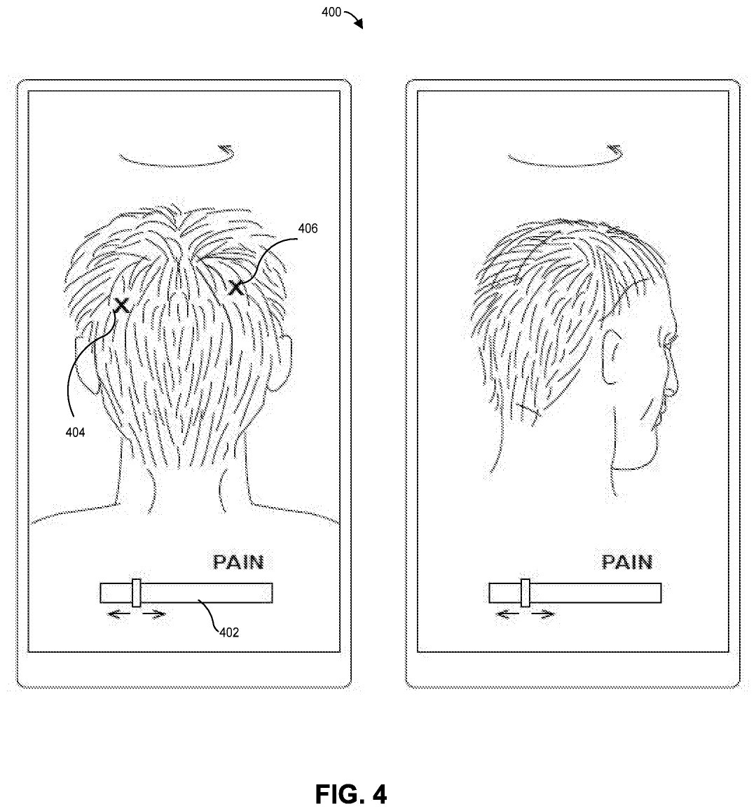

In one example, the user interface 116 can display a screen as illustrated in FIG. 4, through which a user can provide feedback regarding pain experienced during or after the administration of TMS therapy. For instance, the user interface 116 can display an image of a human scalp, which the user can rotate to select specific scalp areas where the user experienced a difference in sensation during the TMS treatment, such as relief from pain and other symptoms during or after a TMS treatment. As a more specific example, the user can select an area of the scalp via the user interface 116 by tapping, a long-press event, etc. and then move a slider displayed via the user interface 116 in one direction to indicate greater relief from pain and in the other direction to indicate lesser relief from pain. In other implementations, the user selects a region of the scalp (e.g., front right quadrant, back left quadrant, etc.) and a numerical (e.g., 0=no decrease in pain, 10=maximum decrease in pain) or qualitative pain (e.g., “reduced pain,” “no change in pain,” etc.) rating from a list via the user interface 116. Moreover, in still other implementations, the user interface 116 provides the user with a first interactive screen prior to treatment, via which he or she can indicate an initial pain level or intensity at one or more respective locations, as well as a second interactive screen after the treatment, via which the user can indicate the pain level or intensity subsequent to the treatment. The TMS application 122 in this manner can generate quantitative metrics indicative of improvement in the user's conditions due to the treatment.

In some examples, the camera 130 captures images or videos of the user to be used for generating user subjective feedback data. In one example, the TMS application 122 analyzes images or videos captured of the user during TMS treatments to identify user facial expressions during these treatments. For example, using a facial recognition algorithm, the TMS application 122 can determine whether a user is likely smiling or laughing (e.g., indicating little to no pain currently being experienced, and/or improvements in various symptoms), or frowning or grimacing (e.g., indicating changes in affect or mood) during TMS treatment.

Additionally, in some examples, the microphone 132 captures audio recordings of the user that are used for generating user data regarding subjective impressions and feelings. In one example, the TMS application 122 analyzes an audio recording captured during a TMS treatment to identify words or sounds originating from the user during the TMS treatment. For instance, using a voice recognition algorithm, the TMS application 122 can identify words spoken by the user during the TMS treatment, and can further determine whether these words likely indicate subjective changes, side effects or relief from symptoms experienced by the user.

In some implementations, the one or more sensors 134 capture indications of motion of the computing device 102 during TMS treatment that can be used for generating user symptom data. For instance, the sensors 134 can include motion sensors, such as a gyroscope, an accelerometer, etc. In one example, the TMS application 122 analyzes data captured by these motion sensors to identify instances in which a user holding the computing device 102 experiences symptoms such as tremors or seizures or decrease in these symptoms.

Referring now to the server 104, generally speaking, the server 104 includes a memory 113 and one or more general-purpose (e.g., microcontrollers and/or microprocessors) or special-purpose processors 117. The memory 113 and one or more processors 117 collectively can be referred to as “processing hardware.” The server 104 is communicatively connected to a prior experience database 108.

The prior experience database 108 can store anonymized data descriptive of prior TMS sessions conducted with other users. This data can originate from the computing device 102 as well as other computing devices associated with other users (not shown). This data can include symptoms or conditions associated with each user and other user information (such as age, gender, etc.) as well as TMS session parameters that were used when TMS therapy was administered to the user. Additionally, this data can include indications of both positive results (e.g., test results indicating treatment of symptoms, positive user feedback) and negative results (e.g., test results indicating worsening of symptoms, negative user feedback such as headache) each user experiences during or after TMS therapy is administered using these parameters.

The memory 113 of the server 104 can be a non-transitory memory and can include one or several suitable memory modules, such as random access memory (RAM), read-only memory (ROM), flash memory, other types of persistent memory, etc. The memory 113 further includes a TMS master application 110, a TMS master ML model 111, and a TMS controller library 115.

Similar to how the TMS application 124 trains and operates the TMS ML model 122, the TMS master application 110 can train and operate the TMS master ML model 111 in accordance with the scheme illustrated in FIG. 3, using data from other users stored in the prior experience database 108. The TMS stimulus parameters generated by the TMS master ML model 111 are then stored in the TMS controller library 115, from which these stimulus parameters can be transmitted to the computing device 102 for use as initial baseline parameters, either generally or by demographic or symptom or disease condition. For example, the computing device 102 may retrieve a set of baseline TMS stimulus parameters for a particular condition and may send instructions to the controller 140 for controlling the TMS device in accordance with these parameters. Using these baseline TMS stimulus parameters and the results thereof, the TMS ML model 122 can be trained to generate TMS stimulus parameters that are specialized for the particular user associated with the computing device 102.

Generally speaking, the TMS device 140 includes control circuitry 142 for controlling one or more microstimulators 144 of a device for providing TMS to an individual. In some implementations, some or all of the one or more microstimulators 144 reside on a motorized antero-posterior and medio-lateral frame, and the control circuitry 142 can reposition microstimulators 144 of the TMS device during operation. The control circuitry 142 also can activate or deactivate microstimulators 144 or change the frequency or pulse parameters of the stimulus, etc., as needed, in order to administer individualized TMS therapy to the user based on instructions received from the computing device 102.

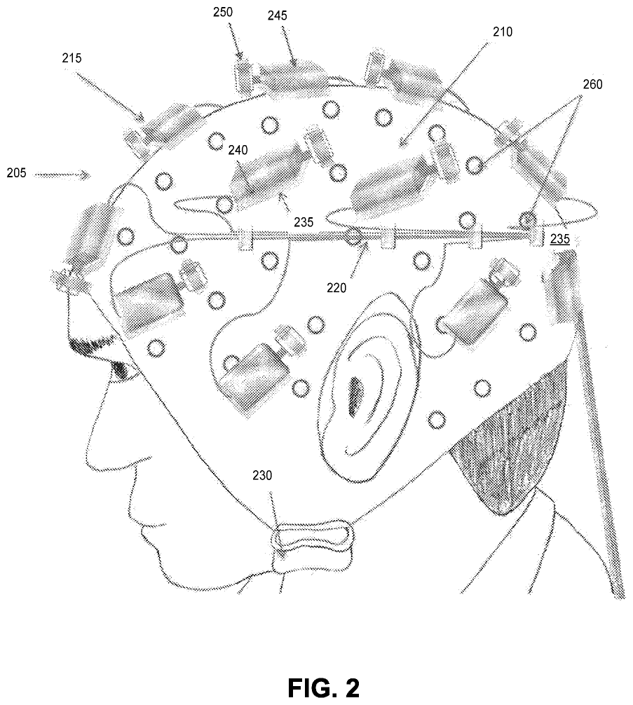

For clarity, an example TMS device with which the TMS application 122 can cooperate is discussed in greater detail with respect to FIG. 2.

An example device 205 for providing TMS to an individual generally comprises a head mount 210 for positioning on the head of an individual, magnet assemblies 215 which are releasably mounted to head mount 210, and leads 220 for connecting each of the magnet assemblies 215 to a computerized controller 225. The computerized controller 225 may be a self-standing device or may be wearable, e.g., on a waistband, an armband, etc. Additionally, in some embodiments, magnet assemblies 215 may be connected to computerized controller 225 wirelessly, whereby to eliminate the need for leads 220.

In one embodiment, the head mount 210 comprises a soft, form-fitting skull cap adapted to cover the head of the individual while leaving the face and ears of the individual exposed. The head mount 210 is intended to provide a stable support for the aforementioned magnet assemblies 215. For example, in one embodiment, head mount 210 comprises a textile construct (e.g., woven, braided or knit fibers) that has a stable structure but which can breathe (for comfort of the individual). Alternatively, in another embodiment, the head mount 210 is constructed of other materials such as soft plastic. The head mount 210 may additionally include a chin strap 230 so that the head mount can be fastened onto the head of an individual with light tension, whereby to ensure that the head mount maintains a fixed position on the head of the individual.

As noted above, magnet assemblies 215 are releasably mounted to the head mount 210. More particularly, magnet assemblies 215 are releasably mounted to the head mount 210 so that the number of magnet assemblies 215, and/or their individual positioning on the head mount 210, can be varied as desired by a clinician or an investigator. To this end, the head mount 210 comprises fastener bases 235 which are distributed about the outer surface of head mount 210, and each of the magnet assemblies 215 comprises a counterpart fastener connect 240 adapted to mate with a fastener base 235, whereby to allow each magnet assembly 215 to be releasably secured to head mount 210 substantially anywhere about the surface of the head mount. It will be appreciated that, as a result of this construction, it is possible to releasably secure the desired number of magnet assemblies 215 to the head mount 210, at the desired locations for those magnet assemblies 215, so that the number of magnet assemblies 215, and/or their positioning on the head mount 210, can be varied as desired by the clinician or investigator.

By way of example but not limitation, the head mount 210 may comprise a woven fabric skull cap covering the skull of the individual, the fastener bases 235 disposed on head mount 210 may each comprise one half of a conventional hook-and-loop (e.g., Velcro™) fastener, and the fastener connects 240 of the magnet assemblies 215 may each comprise the second half of a conventional hook-and-loop (e.g., Velcro™) fastener. In this way, each of the magnet assemblies 215 may be releasably fastened to a fastener base 235, and hence to head mount 210. Alternatively, means other than conventional hook-and-loop (e.g., Velcro™) fasteners (e.g., mechanical fasteners, snap fasteners, etc.) may be used to releasably secure magnet assemblies 215 to head mount 210.

In one embodiment, magnet assemblies 215 each comprise a motor 245 and a permanent magnet 250. The permanent magnets 250 are each mounted to the drive shaft 255 of the motor 245, such that when the motor 245 is energized, the permanent magnet 250 will rotate to provide a rapidly changing magnetic field about the magnet assembly. In one embodiment, each of the magnet assemblies 215 comprises a permanent magnet 250 for selectively providing a rapidly changing magnetic field of at least 500-600 Tesla/second corresponding to a magnet movement speed of no less than 400 Hertz. As will be appreciated by those knowledgeable in the field of TMS, by applying this rapidly changing magnetic field of at least 500-600 Tesla/second, corresponding to magnet movement speed of no less than 400 Hertz, to the brain of an individual, weak electric currents can be induced in the neurons of the brain of the individual. These weak electric currents modify the natural electrical activity of the brain of the individual to provide therapy to the individual. Furthermore, the motor 245 is a variable speed motor, such that the permanent magnet 250 may be rotated faster or slower, as desired, whereby to adjust the voltage of the electric currents induced in the neurons of the brain of the individual. In one preferred form of the invention, the permanent magnet 250 comprises a rare earth magnet, e.g., a neodymium magnet.

The TMS apparatus or device 205 also comprises a computerized controller 225 for independently controlling the operation of each of the magnet assemblies 215, i.e., turning motors 245 on or off, regulating the speeds of motor rotation, etc. Leads 220 connect computerized controller 225 to each of the magnet assemblies 215.

Factors such as how many magnet assemblies 215 are mounted to head mount 210, where those magnet assemblies 215 are located on head mount 210, when the permanent magnets 250 of the various magnet assemblies 215 are rotated, and the speed of such rotation each affect the spatial, strength and temporal characteristics of the magnetic field which is generated by TMS apparatus 205. Accordingly, by controlling these factors, the computerized controller 225 can cause the TMS apparatus 205 to provide a magnetic field with spatial, strength and temporal characteristics tailored to provide an individual with individual-specific TMS therapy, to assist in diagnosis, and/or to map out brain function in neuroscience research, in various embodiments.

Now referring to FIG. 3, the TMS application 124 can train and operate the TMS ML model 122 in accordance with the scheme 300. The TMS master application 110 can train and operate the TMS master machine learning model 111 in a generally similar manner, but the TMS master application 110 typically operates a significantly larger training data set, and some of the real-time feedback data that can be used with the TMS ML model 122 may not be applicable to the TMS master machine learning model 111. For simplicity, FIG. 3 refers to the TMS application 122 and the TMS machine learning model 124.

The TMS application 122 can receive various input signals, including EEG waveform data 364, historical data and probability estimates for configurations 340, interactive feedback data 330, and TMS session parameters 320. The probability estimates correspond to the likelihood that a particular configuration (positions of microstimulators and/or stimulation parameters applied to these microstimulators, such as frequency, pulse duty cycle, etc.) produces positive feedback, for example. Generally speaking, the feature extraction functions 302 can operate on at least some of these input signals to generate feature vectors, or logical groupings of parameters associated with a particular instance of observing the results of TMS therapy. For example, the feature extraction functions 302 can generate a feature vector that indicates that, for a particular location of four microstimulators on the scalp {(x1, y1), (x2, y2), (x3, y3), and (x4, y4)} expressed using the 10-20 International EEG electrode system projection, for instance, for a certain strength of the magnetic field B, certain frequency, certain pulse duty cycle, etc. (equal for all microstimulators in this example, but in general individually configurable for each microstimulator), and for a certain duration of the procedure, the result corresponds to a certain numeric value of symptom relief selected by the user, a certain measurement M (e.g., a real-time EEG scan), etc. The results can be used as a set of labels for the feature vector.

For example, to process the EEG waveform data 364, the feature extraction functions 302 can compare the waveform to sets of threshold values to determine, for example, the frequency, peaks and valleys, etc.

The feature extraction functions 302 can further receive real-time user feedback data 330, so that the computing device 102 operates on the TMS ML model 122 in a closed-loop configuration. The user feedback data 330 can include for example visual feedback 331, audio feedback 332, GUI feedback 333, etc.

To process visual feedback 331, the TMS application 122 can obtain a still image or video of the user and analyze the image or video to determine whether a user is likely smiling or laughing, or frowning or grimacing (e.g., indicating changes in affect or mood, or worsening of or relief from symptoms) during TMS treatment. In some examples, the length of time the user spends frowning or grimacing may be related to the user's pain level. To this end, any suitable software techniques may be used to analyze the image of the user's face to identify facial expressions associated with certain emotions or feelings. For example, iMotions Facial Expression Analysis software can be used to identify user facial expressions in some embodiments. Additionally, in some examples, a separate facial expression model can be trained using a convolutional neural network, or CNN.

The TMS application 122 can process the audio feedback 332 by extracting certain keywords from the audio stream. For example, the feature extraction functions 302 can receive an audio stream and extract certain words or noises that express positive (“good,” “nice,” etc.) or negative (e.g., “head hurts,” “feeling discomfort”, grunting or groaning noises) reactions to the TMS therapy. Based on a location associated with the user or stored language settings of the user, the TMS application 122 may be configured to recognize words in languages associated with the user (e.g., positive or negative French words if the user is located in France or has language settings set to “French,” positive or negative German words if the user is located in Germany or has language settings set to “German,” etc.) Moreover, in some examples, the TMS application 122 can process the audio feedback to determine an intensity level of any pain experienced by the user. Moreover, certain words may indicate specific sensations experienced by the user (e.g., certain words can indicate that the user is experiencing flashes of light). As another example, more frequent words or noises associated with negative reactions to the TMS therapy can indicate a higher intensity of some adverse effects experienced by the user. Similarly, louder words or noises associated with negative reactions to the TMS therapy can also indicate a higher intensity of such untoward effects experienced by the user. In some examples, a separate audio feedback model can be trained using suitable machine learning techniques.

To process the GUI feedback 333, the TMS application 122 can pair indications of location and relief from chronic pain, tremor and other symptoms provided by the user via a GUI (e.g., as shown in FIG. 4). For example, a user can select an area of the scalp where he or she is experiencing relief from chronic pain and also select or enter the extent of relief numerically. The TMS application 122 can process this data into a tuple (x, y, z, p), where x, y, and z indicate circular coordinates corresponding to scalp locations and p indicates the degree of benefit.

Accordingly, the feature extraction functions 302 can generate feature vectors 350 using the real-time user feedback data 330 (visual feedback 331, audio feedback 332, and/or GUI feedback 333), and the TMS session parameters 320 (location of stimuli 321, frequency of rotation of the magnet 322, session duration 323, and date and time of session 324) from the TMS session in which each set of real-time user feedback data 330 was obtained.

The TMS application 122 can further receive input signals associated with test results 310 from scans performed in hospitals or medical laboratories, including, for example, MRI scan data 311, PET scan data 312, and EEG waveform data 313. Using these test results 310 and biometric data 315 associated with the user (e.g., indicating size and/or dimensions of the user's head), a target site identification module 317 can determine initial stimulus locations 318 for the user. For example, the test results 310, individually or collectively, may indicate a particular set of symptoms associated with a certain condition. Accordingly, using the test results 310 and the biometric data 315, the target site identification module 317 selects a location of the user's scalp corresponding to an area of the brain associated with the identified condition as an initial stimulus location 318.

As a more specific example, the target site identification module 317 can receive the MRI scan data 311 in the form of an image and execute an algorithm for identifying areas that are likely to be affected by TMS therapy. In an example implementation, the target site identification module 317 implements a convolutional neural network (CNN), trained with a relatively large set of MRI scans. The target site identification module 317 similarly can be trained with a set of PET scans to receive an image of a PET scan and generate predictions regarding effective placement of microstimulators. The target site identification module 317 in general can be trained with data sets that include one, several, or all of the types of inputs 311, 312, and 313.

In general, the TMS application 122 can train the TMS operation model 124 using supervised learning, unsupervised learning, reinforcement learning, or any other suitable technique. Moreover, the TMS application 122 can train the TMS operation model 124 as a standard regression model. Specifically, the TMS application 122 can train the TMS machine learning model 124 using the generated feature vectors 350 and initial stimulus locations 318, along with constraints such as user-specific use restrictions 308, global use restrictions, and default operational parameters 305. Because the TMS application 122 does not modify the inputs 305, 306, 308, and 318 during training, these inputs can be considered hyperparameters.

Over time, as the TMS application 122 trains the TMS machine learning model 124, the TMS operation model 124 can learn to predict TMS stimulus parameters (locations of microstimulators 391, frequency of rotation 392, pulse width 393, pulse duration 394, etc.) associated with positive user feedback.

The TMS application 122 can send the TMS stimulus parameters generated by the TMS machine learning model 124 as instructions to the controller 142. When the TMS device 140 administers TMS therapy in accordance with the generated TMS stimulus parameters, new interactive feedback data 330 can be generated and used in subsequent training of the TMS operation model 124, i.e., for fine-tuning to improve the performance of the TMS operation model.

While the TMS operation model 124 described above is discussed with respect to data from only one individual, in some implementations additional TMS operation models can be trained using data from multiple individuals. In particular, the TMS master application 110 can obtain data from multiple individuals experiencing similar symptoms and use this data to train a TMS master model 111 that can be subsequently used to generate generalized TMS stimulus parameters for treating those specific symptoms. For example, these symptom- or condition- specific TMS stimulus parameters may be used as initial stimulus parameters for a particular user, and then further refined using an individualized TMS operation model 124.

As another example, the techniques discussed above can be used to train a model for generating operational parameters for controlling a medical rehabilitation system that includes an exoskeleton and an associated controller. Similar to the examples above, a computing device such as a personal computer, a tablet computer, a smartphone, or a dedicated electronic device (e.g., a microcontroller embedded in a bracelet or another wearable article) can train a model using a set of initial parameters, hyperparameters, feedback signals, etc. The inputs for an example therapy session can include control signals provided to the various components of the exoskeleton (e.g., various properties of the drive signals motors that operate on the respective joints), electrical signals received from muscle sensors in the affected area (e.g., the area of the injury or amputation), other sensor readings (e.g., an accelerometer, a gyroscope), verbal and non-verbal user feedback indicating discomfort or pain due to a certain maneuver of the exoskeleton, etc. The sensor readings and/or user feedback can indicate whether the movement occurred at the right rate, along the right direction, at the right time, etc. Examples of the hyperparameters in this example can include the user's weight and height. Similar to the TMS operation model 124, the model can dynamically adjust the outputs provided to the motors and/or other active components of the system, as a result of training. In a generally similar manner, these techniques can be applied to a robotic prosthetic limb, for example.

In another example, these techniques could be used to train a model for generating operational parameters for controlling a cochlear implant. In this example, the model can receive the operational parameters which a controller applies to the transmitter and the receiver/stimulator (which receive signals from a microphone within the implant and convert these signals into electric impulses) as well as the operational parameters which the controller applies to the electrode array as the array collects the impulses from the stimulator and sends these signals to different regions of the auditory nerve. The model in some implementations also can receive pre-defined sounds or words played for the user (e.g., from a sample recording defining the ground truth in training), and the user's interpretation of each sound or word as he or she perceives these sounds through the implant. As a more specific example, the model can receive an indication of whether the user heard anything at all, whether the word or sound was too quiet, whether the word or sound was too loud, whether the user heard the correct word or sound. Further, the model can receive additional indications of whether the user hears any background noise (e.g., buzzing, ringing) via the implant. Accordingly, in this example, a cochlear implant operation model can learn user-specific operational parameters over a certain period of time and adjust the operational parameters in order to improve the accuracy of the impulses sent to the auditory nerve in response to various sounds or words.

In some cases, a model can receive real-time feedback from various sensors and/or verbally or non-verbally from the user. In other cases, no reliable real-time feedback can be available, and the system can receive feedback only periodically. As a more specific example, a certain system can use one or several magnetic stimulators to disrupt a mitochondrial function in cancer cells, thereby triggering apoptosis in the cancer cells without disrupting healthy cells, and the feedback may be available only in the form of quantitative indicators which a medical practitioner can provide after analyzing the results of a lab test.

Further, the controller of the medical rehabilitation system, the prosthetic system, or the implant system also can implement the safety features similar to those discussed above. In particular, the controller can ensure the user is properly matched based to the therapy or the implant based on various biometric parameters such as voice recognition, facial recognition, etc. The controller also can ensure that the system operates within the prescribed limits or ranges (e.g., the extent of movement of the exoskeleton, the maximum strength of the electric impulse provided to the auditory nerve, the duration and frequency of the cancer-treatment therapy).

In at least some of the examples discussed above, user-specific models can generate operational parameters for individual users, and a network server can receive anonymized data from multiple user-specific models to continuously train a master model, similar to the TMS master model 111.

Referring to FIG. 4, an example TMS application on a computing device 102 can generate a user interface for collecting user feedback associated with TMS therapy. As shown in FIG. 4, the user interface screen 400 displays a dynamic model of a human scalp which a user can rotate to select areas of increased or decreased pain. For example, a user can select an area of the scalp 404 or 406 and can use a slider 402 as shown on the user interface screen 400 to indicate a pain level associated with that area of the scalp in some embodiments. In some examples, the dynamic model shown via the user interface screen 400 may include additional or alternative body parts (e.g., a full body model, a model of an arm or a leg, etc.). Accordingly, a user can rotate any body parts shown to indicate areas of increased or decreased pain. Additionally, while the example user interface screen 400 displays a slider feature 402 that a user can use to indicate a pain level or degree of relief from pain, the user interface screen 400 could include additional or alternative means for rating a pain level or degree of relief from pain. For instance, a user could select or enter a number indicating the user's pain level or degree of relief from pain in some embodiments. In particular, the user can use the user interface screen 400 to indicate an initial location and level or intensity of pain or other symptoms/conditions present prior to treatment, and indicate the locations and intensity level of pain or other symptoms/conditions present after treatment.

Additionally, in some instances, the computing device 102 is configured to detect voice input from the user identifying the location and/or degree of relief from pain or other symptoms. In any case, the user feedback collected via the computing device 102 can be used as user GUI feedback 333 in training the TMS operation model 124, as discussed above with respect to FIG. 3.

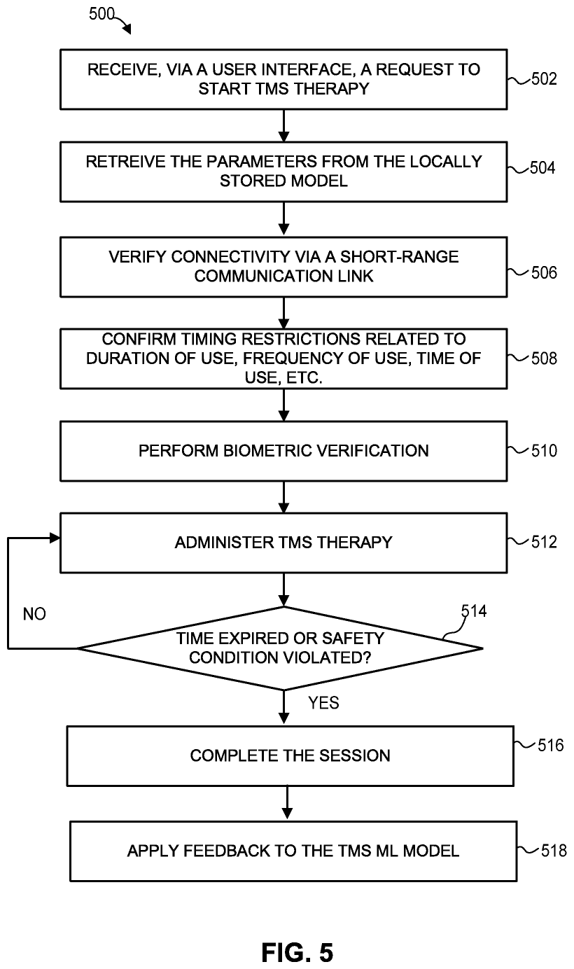

Several example methods that can be implemented in the system of FIG. 1 are discussed next with reference to FIGS. 5, 6, and 7. Referring first to FIG. 5, an example method 500 for controlling the provision of TMS therapy to an individual can be implemented as a set of instructions stored on a computer-readable memory and executable by one or more processors of the computing device 102.

At block 502, the computing device 102 receives a request (e.g., via the user interface 116), indicating that a user of the computing device 102 is attempting to start TMS therapy. For example, the user interface 116 may present a button for selecting an option of starting TMS therapy, and the user of the mobile device 102 may select the button. Alternatively, the user of the mobile device 102 may attempt to start TMS therapy via a voice command, or by other suitable means.

At block 504, the computing device 102 retrieves TMS stimulus parameters from a locally stored model. These TMS stimulus parameters may include, for example, locations of stimulus, frequencies of magnet rotation, stimulus duration, etc. In particular, these TMS stimulus parameters may be individualized for the user of the computing device 102.

At block 506, the computing device 102 verifies its connectivity to a TMS device 140 is verified via a short range communication link (e.g., Bluetooth), i.e., indicating that the user of the computing device 102 is nearby the TMS device 140 that is to be used to administer TMS therapy.

At block 508, the computing device 102 confirms timing restrictions related to frequency of use of the TMS device 140, duration of use of the TMS device 140, etc. These timing restrictions may be global or they may be specific to the user. In any case, these timing restrictions ensure that the user receives the correct frequency or duration of TMS treatment for his or her condition or symptoms, and ensure that the frequency and duration of TMS treatment is safe for the user.

At block 510, the computing device 102 performs a biometric verification. In one example, the computing device 102 captures an image of the user via a camera 130 and attempts to identify the user of the computing device 102 based on the captured image, e.g., using a facial recognition algorithm to compare the captured image to stored images of the user. By accurately confirming the identity of the user of the computing device 102 before allowing the user to begin TMS treatment controlled by instructions from the computing device 102, the method 500 ensures that the user of the computing device 102 receives a TMS treatment that is safe and is tailored to his or her individual needs.

At block 512, the computing device 102 transmits instructions to the controller 142 of the TMS device 140 via the short range communication link in order to administer TMS therapy in accordance with the retrieved TMS stimulus parameters. For example, the instructions can cause the control circuitry 142 to reposition microstimulators 144 of the TMS device, activate or deactivate microstimulators 144 of the TMS device in certain locations, change the frequency of rotation, pulse duty cycle, pulse duration, etc., as needed, in order to administer individualized TMS therapy to the user.

At block 514, the computing device 102 determines whether the time for the TMS therapy session has expired or whether the TMS therapy session has violated any safety conditions. In some examples, user feedback can indicate a violated safety condition (e.g., if the user feedback indicates that a user is experiencing persistent muscle twitching or sensory after-effects). If not (block 514, NO), the computing device 102 continues to transmit instructions to the controller 142 of the TMS device to administer TMS therapy. If either the time has expired or the TMS therapy session has violated a safety condition (block 514, YES), the method proceeds to block 516.

At block 516, the computing device 102 completes the TMS therapy session by ceasing instructions to the controller 142 of the TMS device 140 (or by transmitting instructions causing the controller 142 of the TMS device 140 to stop the TMS therapy). At block 518, the computing device 102 provides any user feedback collected during or after the TMS session, as well as the TMS session parameters, to the machine learning model 122 for training.

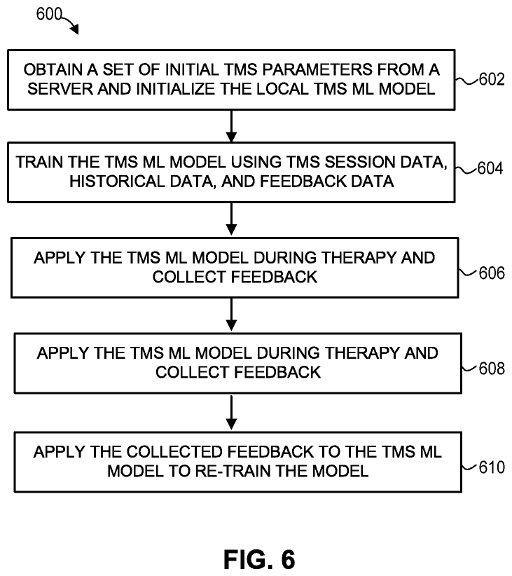

Referring now to FIG. 6, an example method 600 for controlling the provision of TMS therapy to an individual can be implemented as a set of instructions stored on a computer-readable memory and executable by one or more processors of the computing device 102.

At block 602, the computing device 102 obtains a set of initial parameters from a server and initializes a TMS machine learning model using these initial parameters. For example, these initial parameters may be parameters derived from test results (e.g., MRI scan data, PET scan data, EEG waveform data, etc.). At block 604, the computing device 102 trains the TMS machine learning model using TMS session parameters, historical TMS data, and user feedback data obtained during or after TMS sessions.

At block 606, the computing device 102 applies the TMS machine learning model to generate TMS stimulus parameters to be used in administering a session of TMS therapy, and collects user feedback during or after the session. At block 608, the computing device 102 again applies the TMS machine learning model to generate TMS stimulus parameters to be used in administering a session of TMS therapy, and collects user feedback during or after the session. At block 610, the computing device 102 applies the feedback collected at blocks 606 and 608 to the TMS machine learning model to re-train the model.

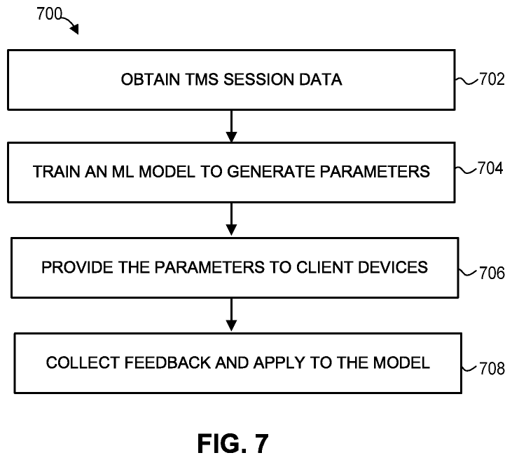

Referring now to FIG. 7, an example method 700 for controlling the provision of TMS therapy to an individual can be implemented as a set of instructions stored on a computer-readable memory and executable by one or more processors of the computing device 102.

At block 702, the computing device 102 obtains TMS session data originating from client devices associated with various users. At block 704, the computing device 102 trains a machine learning model using the TMS session data from the various users and uses the machine learning model to generate TMS stimulus parameters.

At block 706, the computing device 102 provides the TMS stimulus parameters to the client devices. Generally speaking, the client devices control TMS devices to administer TMS therapy using the provided TMS stimulus parameters as initial or baseline TMS stimulus parameters, and users of each client device provide feedback during or after the administered TMS therapy. At block 708, the computing device 102 collects the user feedback associated with the TMS sessions controlled by the various client devices, and trains the machine learning model using the collected feedback.

### Additional Considerations

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter of the present disclosure.

Additionally, certain embodiments are described herein as including logic or a number of components, modules, or mechanisms. Modules may constitute either software modules (e.g., code stored on a machine-readable medium) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term hardware should be understood to encompass a tangible entity, be that an entity that is physically constructed, permanently configured (e.g., hardwired), or temporarily configured (e.g., programmed) to operate in a certain manner or to perform certain operations described herein. As used herein “hardware-implemented module” refers to a hardware module. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may be accordingly configured on a processor, for example, to constitute a particular hardware module at one instance of time and to constitute a different hardware module at a different instance of time.

Hardware modules can provide information to, and receive information from, other hardware. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The methods 500 and 600 may include one or more function blocks, modules, individual functions or routines in the form of tangible computer-executable instructions that are stored in a non-transitory computer-readable storage medium and executed using a processor of a computing device (e.g., a server, a personal computer, a smart phone, a tablet computer, a smart watch, a mobile computing device, or other personal computing device, as described herein). The methods 500 and 600 may be included as part of any backend server, portable device modules of the example environment, for example, or as part of a module that is external to such an environment. Though the figures may be described with reference to the other figures for ease of explanation, the methods 500 and 600 can be utilized with other objects and user interfaces. Furthermore, although the explanation above describes steps of the methods 500 and 600 being performed by specific devices, this is done for illustration purposes only.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as an SaaS. For example, as indicated above, at least some of the operations may be performed by a group of computers (as examples of machines including processors), these operations being accessible via a network (e.g., the Internet) and via one or more appropriate interfaces (e.g., APIs).

Still further, the figures depict some embodiments of the example environment for purposes of illustration only. One skilled in the art will readily recognize from the following discussion that alternative embodiments of the structures and methods illustrated herein may be employed without departing from the principles described herein.

Upon reading this disclosure, those of skill in the art will appreciate still additional alternative structural and functional designs for controlling the provision of TMS therapy to an individual through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

### Aspects

Embodiments of the techniques described in the present disclosure may include any number of the following aspects, either alone or combination:

1. A computer-implemented method for generating operational parameters for Transcranial Magnetic Stimulation (TMS), the method comprising: generating, by the processing hardware, training data that includes indications of previously conducted TMS sessions; training, by the processing hardware, a machine learning model using the training data; applying, by the processing hardware, the trained machine learning model one or more parameters related to an individual to generate operational parameters for a TMS session; and causing, by the processing hardware, a device for applying TMS to conduct a TMS session with the individual in accordance with the generate operational parameters.

2. The method of aspect 1, wherein generating the training data includes receiving, for each of the previously conducted TMS sessions, indications of one or more of: (i) a duration of the TMS session, (ii) respective locations on scalp of a plurality of stimulators, or (iii) operational parameters applied to the plurality of stimulators.

3. The method of aspect 2, wherein the operational parameters include a frequency of rotation of a magnet.

4. The method of any of aspects 2 or 3, wherein the operational parameters include characteristics for an electric signal applied to the corresponding stimulator.

5. The method of any of aspects 1-4, further comprising: receiving, by the processing hardware, real-time feedback from the individual during the session, generating, by the processing hardware, one or more quantitative metrics based on the real-time feedback, and applying the one or more quantitative metrics to the machine learning model to further train the machine learning model.

6. The method of aspect 5, wherein receiving the real-time feedback includes receiving, via an interactive user interface, a location on a scalp at which the individual experienced side effects or relief from pain or a disease-specific symptom during the TMS session.

7. The method of aspect 6, wherein receiving the real-time feedback further includes receiving, via the interactive user interface, an indication of intensity of the pain or a disease-specific symptom.

8. The method of any of aspects 5-7, wherein receiving the real-time feedback includes receiving, via an audio input device, an indication of whether the individual experienced side effects or relief from pain or a disease-specific symptom during the TMS session.

9. The method of any of aspects 5-8, wherein receiving the real-time feedback includes detecting, using a camera, a facial expression indicative of subjective experiences, during the TMS session.

10. The method of any of aspects 5-9, wherein receiving the real-time feedback includes receiving an encephalogram (EEG) reading.

11. The method of any of aspects 1-10, further comprising: receiving an initial set of locations on a scalp for placing a plurality of stimulators of the device for applying TMS.

12. The method of aspect 11, further comprising: receiving test data including at least one of a magnetic resonance imaging (MRI) scan, a positron emission tomography (PET) scan, or an EEG reading; receiving biometric data for the individual; and generating the initial set locations on the scalp using the test data and the biometric data.

13. A computing device comprising: one or more processors; a user interface; an interface to couple the computing device to a device for applying TMS to an individual during a TMS session; and a computer-readable memory coupled to the one or more processors, the memory storing instructions that implement a method according to any of aspects 1-12.

14. A method in a computing device for controlling application of a TMS therapy to an individual, the method comprising: receiving, via a user interface, a request from a user to start TMS therapy; detecting, by processing hardware, a short-range communication link between the computing device and a controller of a device for applying TMS therapy to the user during a TMS session; verifying, by the processing hardware, identity of the user; and transmitting, by the processing hardware via the short-range communication link, a set of instructions for the controller to conduct the TMS session in accordance with the verified identity of the user.

15. The method of aspect 14, wherein verifying the identity of the user includes obtaining, by the processing hardware, biometric data for the user.

16. The method of aspect 15, wherein the obtaining the biometric data for the user includes: capturing an image of the user's face, and applying a facial recognition function to identify the user.

17. The method of any of aspects 14-16, wherein the transmitted set of instructions specifies one or more of: (i) a duration of the TMS session, (ii) respective locations on scalp of a plurality of stimulators, or (iii) operational parameters applied to the plurality of stimulators.

1. A system for administering dynamically adjustable Transcranial Magnetic Stimulation (TMS) treatment, the system comprising:
a TMS device including:
a control circuitry, and
a plurality of stimulators coupled to the control circuity and configured to generate a changing magnetic field; and
a computing device including:
one or more processors, and
a peripheral interface for communicating with the TMS;
the computing device configured to:
provide, via the peripheral interface, first control signals to the TMS device to conduct TMS session in accordance with an initial set of TMS operational parameters;
receive feedback related to the TMS session;
automatically modify the initial set of TMS operational parameters based on the received feedback to generate modified TMS operational parameters; and
provide second control signals to the TMS device to conduct the TMS session in accordance with the modified TMS operational operators.

2. The system of claim 1, wherein:
the TMS device further comprises a motorized frame on which the plurality of stimulators are mounted; and
the first and second control signals include indications of initial and modified positions, respectively, for the plurality of stimulators.

3. The system of claim 2, wherein the frame is an antero-posterior or a medio-lateral.

4. The system of claim 1, wherein:
each of the plurality of stimulators includes a motor configured to rotate a magnet to provide a rapidly changing magnetic field; and
the first and second control signals include indications of initial and modified speeds of rotation, respectively, for the plurality of stimulators.

5. The system of claim 1, wherein the computing device is further configured to:
receive training data that includes indications of previously conducted TMS sessions;
train a machine learning model using the training data; and
modify the initial set of TMS operational parameters to generate the modified TMS operational parameters by applying the trained machine learning model to the initial set of TMS operational parameters and the received feedback.

6. The system of claim 5, wherein the computing device is configured to receive the training data from a network server configured to receive data indicative of TMS sessions from independent respective systems for administering dynamically adjustable TMS treatment.

7. The system of claim 5, wherein the training data includes, for each of the previously conducted TMS sessions, indications of one or more of:
(i) a duration of the TMS session,
(ii) respective locations on a scalp of the plurality of stimulators during the TMS session,
(iii) characteristics of respective electric signals applied to the plurality of stimulators during the TMS session, or
(iv) feedback regarding the TMS session.

8. The system of any of the preceding claims claim 1,
the computing device further includes a user interface; and
the computing device is configured to receive the feedback related to the TMS session via the user.

9. The system of claim 8, wherein the feedback includes an indication of a location on a scalp at which a subject of the TMS session experienced a difference in sensation during the TMS session.

10. The system of claim 8, wherein the feedback includes an indication of intensity of the pain or a disease-specific symptom at a corresponding location.

11. The system of claim 1, wherein the feedback includes audio input indicative of whether the individual experienced side effects or relief from pain or a disease-specific symptom during the TMS session.

12. The system of claim 1, wherein the feedback includes an encephalogram (EEG) reading.

13. The system of claim 1, wherein the computing device is further configured to:
receive test data including at least one of a magnetic resonance imaging (MRI) scan, a positron emission tomography (PET) scan, or an EEG reading;
receive biometric data for the individual; and
generate an initial set of locations on the scalp using the test data and the biometric data.

14. The system of claim 1, wherein the computing device is further configured to:
receive biometric data from a user, and
verify identity of the user using the biometric data to identify the set of initial set of TMS operational parameters specific to the user.

15. A method, for administering dynamically adjustable Transcranial Magnetic Stimulation (TMS) treatment, the method comprising:
providing, by one or more processors, first control signals to a TMS device, the TMS device including a control circuitry, and a plurality of stimulators coupled to the control circuity and configured to generate a changing magnetic field to conduct a TMS session in accordance with an initial set of TMS operational parameters;
receiving, by the one or more processors, feedback related to the TMS session;
automatically modifying, by the one or more processors, the initial set of TMS operational parameters based on the received feedback to generate modified TMS operational parameters; and
providing, by the one or more processors, second control signals to the TMS device to conduct the TMS session in accordance with the modified TMS operational operators.

16. The method of claim 15, further comprising:
receiving, by the one or more processors, training data that includes indications of previously conducted TMS sessions;
training, by the one or more processors, a machine learning model using the training data; and
modifying, by the one or more processors, the initial set of TMS operational parameters to generate the modified TMS operational parameters by applying the trained machine learning model to the initial set of TMS operational parameters and the received feedback.

17. The method of claim 16, wherein receiving the training data that includes indications of previously conducted TMS sessions includes receiving the training data from a network server configured to receive data indicative of TMS sessions from independent respective systems for administering dynamically adjustable TMS treatment.

18. The method of claim 16, wherein the training data includes, for each of the previously conducted TMS sessions, indications of one or more of:
(i) a duration of the TMS session,
(ii) respective locations on a scalp of the plurality of stimulators during the TMS session,
(iii) characteristics of respective electric signals applied to the plurality of stimulators during the TMS session, or
(iv) feedback regarding the TMS session.

19. The method of claim 15, further comprising:
receiving, by the one or more processors, test data including at least one of a magnetic resonance imaging (MRI) scan, a positron emission tomography (PET) scan, or an EEG reading;
receiving, by the one or more processors, biometric data for the individual; and
generating, by the one or more processors, an initial set of locations on the scalp using the test data and the biometric data.

20. The method of claim 15, further comprising:
receiving, by the one or more processors, biometric data from a user, and
verifying, by the one or more processors, an identity of the user using the biometric data to identify the set of initial set of TMS operational parameters specific to the user.
